(19) United States
(12) Patent Application Publication
(10) Pub. No.: US 2006/0029633 A1
(43) Pub. Date: Feb. 9, 2006

(54) Biological patch for use in medical procedures

(75) Inventors: Ryan A. Kaiser, Leesburg, IN (US); Kevin T. Stone, Winona Lake, IN (US); Troy M. Walters, Plymouth, IN (US); Karen Troxel, Warsaw, IN (US); Frank O. Bonnarens, Prospect, KY (US)

Correspondence Address: HARNESS, DICKEY & PIERCE, P.L.C., P.O. BOX 828, BLOOMFIELD HILLS, MI, 48303, US

(73) Assignee: Arthrotek, Inc, Warsaw, IN (US)

(21) Appl. No.: 10/910,796

(22) Filed: Aug. 3, 2004

(51) Int. Cl. A61F 13/00

(52) U.S. Cl. 424/422

(57) ABSTRACT

A method for forming a patch using a laminated material and a process for forming the laminated material. Also a plurality of patches using the laminated material for various implantation purposes into an anatomy. In addition, an implant method for implanting one or a plurality of the selected patches for various reasons.

### FIELD

The present invention relates to various anatomical implant portions, and particularly to implants for soft tissue to substantially mimic anatomical tissue.

### BACKGROUND

In an anatomy, such as a human anatomy, various portions are generally interconnected with one another to perform selected functions. For example, ligaments interconnect selected bones to hold a selected shape to provide for a selected articulation. Tendons interconnect tissue, such as muscle, with bones to provide anchors for the muscles to allow for the muscles to move selected bone portions. Although in an uninjured anatomy, the various soft tissues are generally able to perform their selected tasks, for various reasons, selected soft tissues may become injured or inoperable. Therefore, the bone portions, connected by ligaments, and the muscle portions, interconnected with the tendons, are not able to perform their selected anatomical functions.

After an injury or event that causes an inoperability or damage to various soft tissues, implants may be provided to perform similar functions in the soft tissue. For example, any graft, such as including a bone-tendon-bone (BTB) graft, can be used to interconnect two bone portions. Generally, the BTB graft can be harvested from a donor site including a selected length of the tendon and two bone portions, one at each end. This BTB graft can then be positioned in a selected anatomical position to replace or repair an injured or damaged anatomical portion. The BTB graft may be taken from the patient to form an autograft.

Alternatively, or in addition to the autograft, it may be desirable to provide an artificial implant that can simulate an anatomical implant. Particularly, to replace the donor site of the graft, such as the BTB graft, it may be desirable to provide a graft of similar material to reinforce the donor site. Therefore, although an autograft can be used, the donor site may be substantially maintained in its natural or strong state. Alternatively, if an appropriate donor site is not found, or it is more preferable to use a xenograft, it may be desirable to provide a xenograft of similar strength and including an appropriate shape.

Regardless, a soft tissue may become injured and allow for its reinforcement or replacement by a soft tissue implant. The soft tissue implant may be able to assist in restoring substantially natural movement and strength to the injured site after the implant.

### SUMMARY

An anatomical implant that can be used to replace various anatomical portions, such as soft tissue including tendons and ligaments is disclosed. The soft tissue implant can be formed of appropriate materials, such as xenograft materials, to substantially mimic a soft tissue. Various materials may be used such as collagen (such as intestinal collagen (ICL)) or human, porcine, or bovine pericardium, dermis, submucosa, etc. These materials can be used to form grafts of appropriate sizes and strengths for implantation into a body to repair or replace injured anatomical tissues. For example, any appropriately shaped graft can be positioned in a tendon that allows for substantial repair of the injury site.

According to an embodiment, a shaped graft may be formed that is substantially complimentary to a donor site for a selected graft. Therefore, a shaped xenograft implant can be positioned in the donor site to provide substantial anatomical or natural strength thereto. The graft may include various features such as preformed suture or anchor sites. Generally, the graft may be formed of a plurality of layers of the xenograft material to form a substantially laminated and strong implant.

Similarly, sheets of the xenograft material can be formed and shaped into appropriate shapes for various implants. For example, an implant of an appropriate shape may repair or replace a scaphoid-lunate ligament and includes anchor sites to allow for a substantially easy implantation and need for no or only minimal surgical customization. Therefore, the graft can be substantially customized and formed preoperatively to minimize time and duration of the surgical procedure.

Nevertheless, a selected graft can be partially or completely cross-linked, such as when the graft is laminated to allow for remolding or a selected rigidity. During the lamination process, the graft may be cross-linked, such as chemically or dehydrothermally, to increase strength and rigidity prior to implantation. In addition, the graft may be positioned on a selected jig or form such that the final graft will include the selected form. The jig or shaper may include various features, such as pins or sharp portions, that both shape and perforate the graft. The perforations may increase surface area to increase and/or allow for cellular ingrowth after implantation. Therefore, the pins may remain in the graft during the lamination or the cross-linking procedure such that appropriate pores remain in the graft.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of a tissue patch or implant according to a first embodiment;

FIG. 2 is a partial cross-sectional perspective view of the patch of FIG. 1 in an implanted position;

FIG. 3 is a perspective view of the patch according to an alternative embodiment;

FIG. 4 is a planned view of a patch according to a further alternative embodiment;

FIG. 5 is an exemplary application of the patch illustrated in FIG. 3;

FIG. 6 is a side view of a patch according to a further alternative embodiment;

FIG. 7 is a top perspective view of the patch of FIG. 6;

FIG. 8 is a top plan view of a patch according to a further alternative embodiment;

FIG. 9 is a end view of a blank stack to form a selected patch;

FIG. 10 is a patch according to FIG. 1 that has been cut from a blank stack;

FIG. 11 is a jig for formation of a selected patch;

FIG. 12 is a selected patch positioned relative to the jig of FIG. 11 to form a selected patch;

FIG. 13 is a detailed view of a shoulder portion including a torn rotator cuff;

FIG. 14 is a detailed view of the shoulder and rotator cuff illustrating an implant procedure according to an embodiment; and

FIG. 15 is a detailed view of a shoulder and a rotator cuff illustrating an implant procedure according to an alternative embodiment.

### DETAILED DESCRIPTION OF THE VARIOUS EMBODIMENTS

The following description of various embodiments is merely exemplary in nature and is in no way intended to limit the invention, its application, or uses.

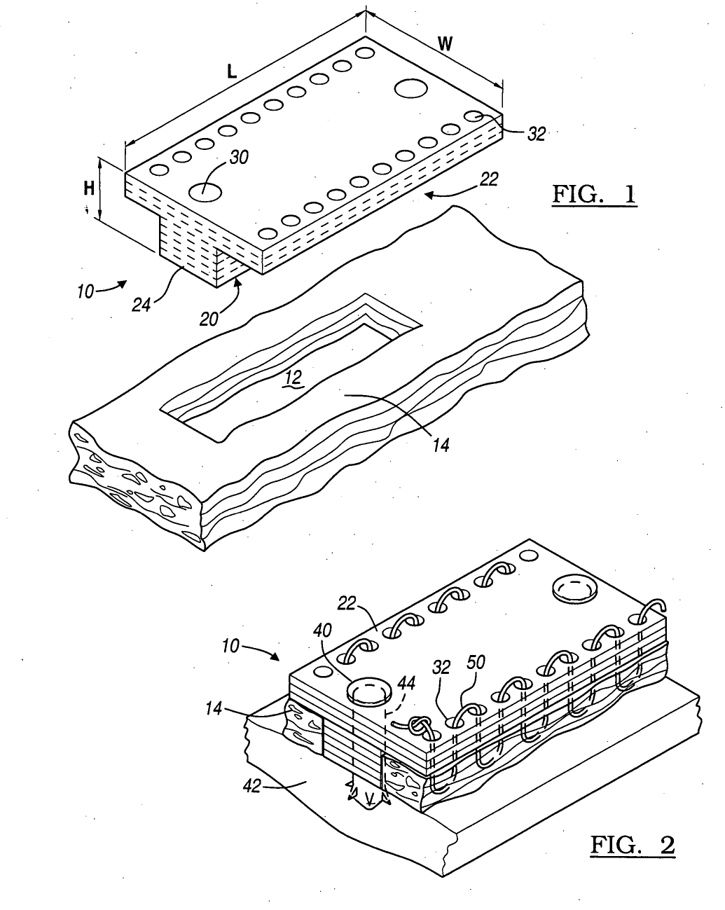

With reference to FIG. 1, a soft tissue implant or patch 10 is illustrated. The patch 10 can be formed in any general shape. For example, the patch 10 can be formed in a substantially T-shape or any other appropriate shape. The T-shape generally has a length L that may be substantially greater than a width W or a height H. The patch 10 is generally formed to fit within or be complimentary with a patch site 12 that is formed in a tissue area 14. The tissue 14 may be any appropriate selected tissue that requires or is selected to include the patch 10. The tissue 14 may be either an autograft or a xenograft from a donor site of an implant that is taken, such as for a BTB graft. Alternatively, the patch site 12 may include a damaged area formed in the soft tissue 14 that the patch 10 may be used to repair. Regardless, the soft tissue 14 includes the patch site 12 into which the patch 10 may be positioned.

The patch 10 may include any appropriate or selected features. For example, the patch 10 may be formed of a plurality of graft layers 20. Each of the individual graft layers 20 may be an appropriate or selected material that is laminated together to form the patch 10. Various exemplary materials include intestinal collagen, or other appropriate materials, and are discussed in detail herein. Similarly, appropriate techniques may be used to laminate the plurality of the layers 20 to form the graft 10, also discussed herein. It will be understood, however, that the patch 10 may be formed of a single or unitary portion. That is, the graft 10 may be formed of a single piece of material that has been shaped into the appropriate shape to be implanted into the graft site 12. The solid piece of material can also include the various features that are used in the patch 10.

The patch 10, generally defining a T-shape, includes an upper or platform area 22 and a base or leg area 24. Although any appropriate shape may be selected, the T-shape including the upper platform 22 and the leg 24, may allow for the patch site 12 to be substantially filled with the patch 10 while allowing for a substantial surface area to affix the patch 10 to the soft tissue 14. For example, a tack area or aperture 30 may be formed in one or both ends of the patch 10. The tack aperture 30 allows for a selected tack or appropriate implant portion to pass through and engage the patch 10 and engage a selected portion of the soft tissue 14 or a underlayment thereof. For example, the soft tissue 14 may be formed around a hard tissue, such as a bone portion. Therefore, the patch area 12 may expose a selected area of the bone to which a tack may be affixed. The tack aperture 30 allows for a tack to pass through the patch 10 to engage the hard bone.

In addition to the tack aperture 30, or in lieu thereof, a plurality of suture bores or apertures 32 may also be provided along the platform area 22. The suture apertures 32 are preformed areas for a suture to pass through the patch 10 and engage the soft tissue 14. In this way, the patch 10 can be quickly affixed to the patch area 12 without substantial intraoperative planning. The patch 10 may be positioned in the patch area 12 and the various apertures, such as the tack aperture 30 or the suture aperture 32 may be used to affix the patch 10 to the selected tissue 14 and the patch area 12.

With reference to FIG. 2, a tack 40 can pass through the tack aperture 30 to engage a bone 42 which is below the soft tissue 14. The tack 40 may include any appropriate portions to engage the bone 42, such as a head 44 that is able to substantially engage the platform area 22 of the patch 10. Therefore, the patch 10 can be securely fixed to the bone 42 so that the patch 10 does not move or become dislocated from the selected position. In addition to the tack 40, a suture 50 can be used to engage the suture apertures 32 to the soft tissue 14. The suture 50 allows for the patch 10 to be fixed to the soft tissue 14 in a selected manner. For example, the suture 50 may crisscross the patch or only be positioned along a length thereof. Nevertheless, the suture 50 allows the patch 10 to be substantially secured to the soft tissue 14 to hold the soft tissue relative to the patch 10 in a selected orientation or position. Therefore, the soft tissue 14 is able to maintain a selected shape assisted by the implantation of the patch 10. The patch 10 may be implanted for any appropriate number of reasons such as an injury to the soft tissue 14 or the removal of an autograft from the patch site 12. Nevertheless, the suture 50 assists in holding the remaining soft tissue 14 in the selected position such that an appropriate healing or reconstruction of the soft tissue 14 may occur.

The patch 10 may be formed of any appropriate material, such as acellular collagen, which can be formed from appropriate tissue, such as intestinal collagen (ICL). These various materials are generally bio-absorbable such that over a selected period of time the patch 10 may be absorbed into the body and the soft tissue 14 may replace it and the patch site 12 may be substantially regrown with the soft tissue 14. Therefore, the patch 10 allows for the patch site 12 to be substantially regrown with natural soft tissue 14 to increase the longevity of the donor site and the patient.

Furthermore, the selected materials allow for a substantially infinite number of appropriate shapes and sizes of the patch 10. The patch 10 may be formed in any appropriate shape or size depending upon the patch site 12. If a larger graft must be taken from the individual then the patch 10 may be formed larger. Also, the patch 10 can be formed preoperatively in any appropriate size or shape for significantly quick implantation during the operative procedure. Nevertheless, the patch 10 may also be molded intraoperatively, at least to a selected degree, to allow for a substantially precise fit of the patch 10 into the patch site 12. Therefore, it may be that the donor site is substantially less traumatized than if no or a non-customized patch were used to repair the donor site.

If a plurality of the layers 20 are used to form the patch 10, the layers may be laminated to form a graft of a selected shape, strength and size. In addition the various layers 20 of the patch 10 may be cross-linked to form a graft of a selected rigidity, strength, and resilience. The cross-linking of the various layers or the material within the various layers 20 may be of a selected amount to select various attributes of the patch 10. For example, a selected amount of cross-linking may be provided such that the patch 10 is substantially moldable during the implant procedure. Alternatively, a higher level of cross-linking may substantially stiffen and make rigid the patch 10 such that the patch 10 is substantially immobile after the cross-linking occurs. Therefore, the various amounts of cross-linking can be selected to achieve a selected property of the patch 10. If a greater rigidity and stiffness of the patch 10 is required, a greater amount of cross-linking may be performed to provide for a substantially stiff and rigid patch 10.

Without being limited by the theory, one use for the patch 10 is to reinforce a donor site for a BTB autograft. In this case, the patch site 12 would be a BTB graft harvest site in which the patch 10 would be used to substantially reinforce. Therefore, the patch 10 may be formed of a selected size to substantially compliment the size of the BTB graft that is harvested from the patch site 12. In this case, the patch 10 may help to reinforce the soft tissue from which the BTB graft is harvested to allow for an increased strength of the donor site, particularly for an autograft procedure. Therefore, the patch 10 may be able to provide for a substantially larger or stronger BTB graft to be removed from an autograft position or from a harvest site to allow greater reinforcement of a second site or injury site. Nevertheless, the patch 10 may also be used as a primary implant or as a replacement for an injured portion of the anatomy rather than simply providing a strengthening or BTB harvest site patch.

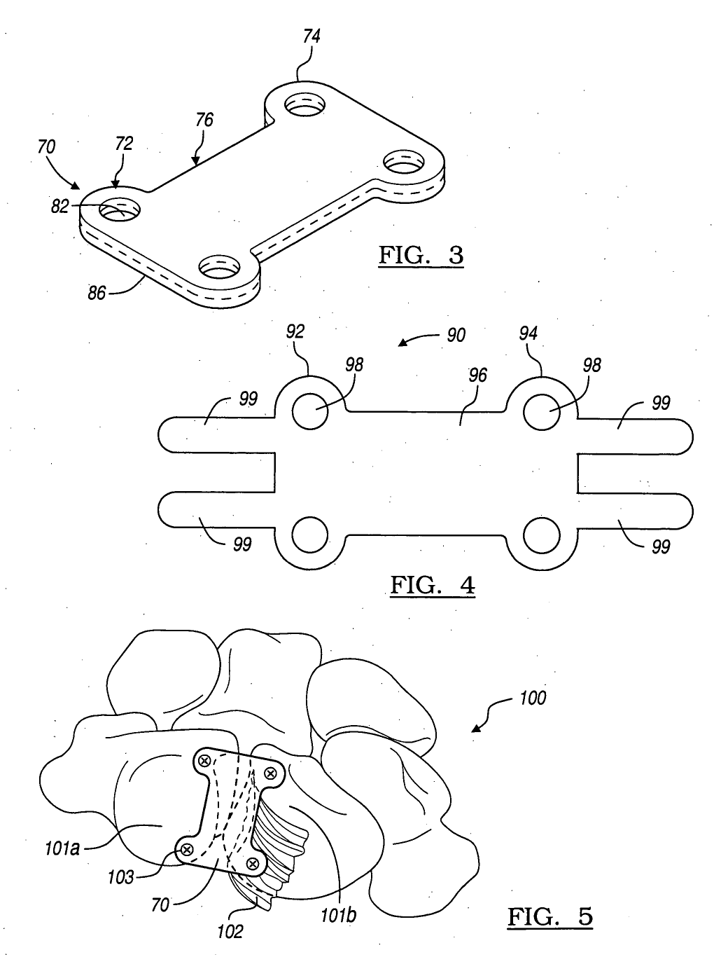

With reference to FIG. 3, a scaphoid-lunate patch 70, similar to the patch 10 in makeup may be formed in a selected shape, such as one to be used in a scaphoid-lunate. The scaphoid patch 70 may be particularly useful in the repairing or reinforcing the a tendon or ligament near the scaphoid or lunate. The scaphoid patch 70 may be used to strengthen or replace a tendon that passes near the scaphoid and lunate through the carpal tunnel to interconnect the metacarpals with the muscle groups in the arm. In addition, the patch 70 may be used to reinforce the soft tissue that surrounds the carpal tunnel for treatment regarding injuries of the carpal tunnel area. Although it will be understood that the scaphoid patch 70 may be formed in any appropriate shape, generally a “dumbbell” or “bone” shape allows for a easy or quick implantation into the scaphoid-lunate during a selected procedure. Generally, the patch 10 includes a first end region 72 and a second end region 74 that is interconnected with a middle portion 76. The various portions allow for a implantation into a selected area to reinforce the selected area.

The scaphoid patch 70 includes at least a first aperture 80. The aperture 80 extends from a first surface to a second surface of the scaphoid patch 70 and allows for fixation of the scaphoid patch 70 to a selected position. The aperture 80 may be used for any appropriate fixation of the scaphoid patch 70 to a selected position. For example, a suture or other fixation mechanism may be passed through the aperture 80 to fix the patch 70 to a selected portion of the soft tissue. In addition, an anchor may be passed through the aperture 80 to anchor the scaphoid patch 70 to a selected soft tissue portion or to a bone portion. It will be understood that either or both a suture and an anchor may be used to fix the scaphoid patch 70 to a selected position. In addition, a particular suture or anchor is not necessary and any appropriate suture or anchor may be used.

The scaphoid patch 70 may also be formed using a layer 84 or a plurality of the layers 84 to form the scaphoid patch 70. As described above, the plurality of the layers 84 may include any appropriate material such as ICL to form the scaphoid patch 70. The plurality of the layers may be laminated together and cross-linked for various characteristics. Nevertheless, it will be understood that the scaphoid patch 70 may also be formed of a substantially single portion of material as a unitary piece. As discussed above, the amount of cross-linking may be used to selectively stiffen or leave moldable the scaphoid patch 70. For example, substantially little cross-linking may be provided to allow for a maximum amount of moldability of the scaphoid patch 70 during the operative procedure.

With reference to FIG. 4, a scaphoid patch 90 similar to the scaphoid patch 70 is illustrated. The scaphoid patch 90 includes enlarged regions 92 and 94 at either end of a central portion 96. Formed within the enlarged regions 92, 94 is an aperture or bore 98 or a plurality of apertures 98. The scaphoid patch 90, however, differs from the scaphoid patch 70 by including at least a finger 99 extending from at least one of the enlarged regions 92, 94. In addition, a plurality of the fingers 99 can be formed to extend longitudinally from the enlarged regions 92, 94 and generally in line with and parallel to the central region or interconnecting region 96. The fingers 99, that extend from the enlarged regions 92, 94 may assist in fixing the patch 90 to the selected region of the anatomy. For example, the fingers may be used to further surround a selected soft tissue portion or interconnect a plurality of regions of the soft tissue with the interconnecting region 96. Therefore, the patch 90 may be used to substantially interconnect or provide an implant for a scaphoid-lunate region and further includes the finger portions 99 to reach various other soft tissue portions.

The scaphoid patch 70 and the scaphoid patch 90 can be used to repair or replace tendons or ligaments in any appropriate region. Generally, the scaphoid-lunate region for the wrist, which is susceptible to various injuries, such as sports injuries or chronic motion injuries, can benefit from the implantation of the scaphoid patch 70, 90.

With reference to FIG. 5, a carpal complex 100 may define a portion of an anatomy, such as a human anatomy. The carpal complex 100 may include a plurality of bone portions such as a scaphoid 101a and a lunate 101b. The scaphoid 101a and lunate 101b define two portions of bone in the carpal complex 100 that articulate relative to one another and to a radius (not illustrated) as understood by one skilled in the art. Generally, during various activities of an individual including the carpal complex 100, various stresses are applied to the carpal complex 100 into the scaphoid 101a and lunate 101b.

In addition, various soft tissue portions such as scaphoid lunate ligament 102 may interconnect the scaphoid 101a and lunate 101b. The scaphoid lunate ligament 102 holds the various bone portions relative to one another to allow for an appropriate articulation and resist dislodging the various bones during stressful activities. Nevertheless, for various reasons, the scaphoid lunate ligament 102 may become injured or damaged. When an injury occurs the various bone portions, such as the scaphoid 101a and the lunate 101b may no longer be held relative to one another in a substantially anatomical or proper manner. Therefore, it may be required or selected to re-secure the scaphoid 101a relative to the lunate 101b.

As described above, various portions may be provided of a material that is operable to replace or repair soft tissue portions. For example, the patch 70 (FIG. 3) may be used to repair various portions of the soft tissue. For example, the patch 70 may be used to repair or replace the scaphoid lunate ligament 102. As illustrated in various embodiments, as in FIG. 5, the patch 70 may be positioned in an operative position between the scaphoid 101a and the lunate 101b. The patch 70 may be attached to the soft tissue portion of the scaphoid lunate ligament 102 or directly to the bones of the scaphoid 101a and the lunate 101b.

For example, an anchor 103, or a plurality of the anchors 103, may be provided though the bores 82 formed in the patch 70 to engage the soft tissue or the bones directly. The patch 70 may therefore may replace or substantially repair the soft tissue interconnection of the scaphoid 101a and the lunate 101b. The soft tissue patch 70 may be attached directly to the bone such that reliance on a natural attachment of the scaphoid lunate ligament 102 is not required. Nevertheless, any appropriate attachment may be used to fix the patch 70 in a selected position relative to the scaphoid 101a and the lunate 101b.

Therefore, the patch 70, or any appropriate patch according to various embodiments, may be used to replace small portions or any appropriate size portions of soft tissue. In addition, the patch as according to various embodiments may be used to be affixed directly to bone portions or to soft tissue portions to provide a replacement thereof, as described above. For example, the patch 70 may include a soft tissue anchor or sutures that pass through the bores 82 to engage the remaining portions of the scaphoid lunate ligament 102. Moreover, any appropriate bone anchor may be provided to pass through the patch 70 to engage the bones 101a, 101b. It will be understood that a specific kind of anchor or suture is not required and any appropriate suture or anchor may be used.

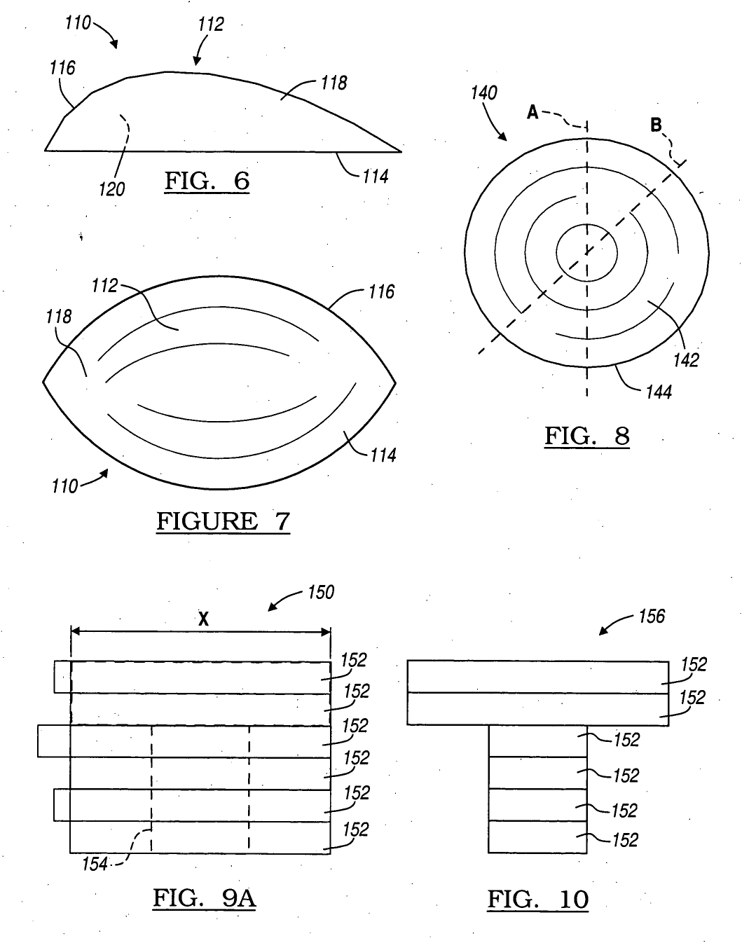

With reference to FIGS. 6 and 7, a rotator cuff implant 110 may be formed to substantially mimic a rotator cuff into which the implant 110 may be implanted. The rotator cuff implant 110 generally includes a radius or dome portion 112 and an edge 114 of a selected appropriate size for the implant 110. The dome portion 112 may be a substantially constant radius or may include a first portion 116 including a smaller radius and a second portion 118 including a larger radius. The multiple radius portions may be customized for selected patients and may differ depending upon the particular patient. Nevertheless, the rotator cuff implant 110 also includes a generally concave interior surface 120 that allows for articulation with a selected anatomical portion. Therefore, the rotator cuff implant 110 may be implanted into a selected anatomical region without substantially forming the rotator cuff implant 110 during the operative procedure.

Nevertheless, it will be understood that the rotator cuff implant 110 may be cut or scribed to allow for a intraoperative customization of the rotator cuff implant 110. Although the rotator cuff implant 110 may be formed prior to implantation, or even the operative procedure, various intraoperative customizations may occur. For example, the rotator cuff patch 110 may be formed using a template that is determined in preoperative planning and may be determined using various x-ray or magnetic resonance imaging techniques to design a selected template. Regardless of customization prior to the operative procedure the rotator cuff patch 110 may require a certain amount of customization during the operative procedure. Therefore, it will be understood, that the rotator cuff implant 110 may be pre-operatively customized, such as including a plurality of radii, and also intra-operatively customized.

With reference to FIG. 8, a substantially constant radius rotator cuff patch 140 is illustrated. The substantially constant radius rotator cuff patch 140 generally includes a radius of a dome region 142 that is substantially constant over the entire surface of the rotator cuff patch 140. Therefore, the rotator cuff patch 140 may be substantially non-patient specific, yet include a dome region 142 to substantially match a general anatomical portion. The substantially constant radius rotator cuff patch 140 may then be cut along a selected plane, such as plane A or plane B, at the time of implantation. It will be understood that any appropriate plane may be cut, and planes A and B are merely exemplary. Therefore, the constant radius rotator cuff patch 140 may be supplied for the operative procedure and an appropriate cut made to form the substantially constant radius rotator cuff patch 140 for the selected patient.

Therefore, a rotator cuff patch 110, 140 may be provided for an operative procedure to replace or repair a rotator cuff of a selected patient. However, a substantially customized rotator cuff patch 110 may be provided for a selected patient if the specifics of the implant required are known prior to the operative procedure, such as with various pre-operative planning techniques. Alternatively, the general rotator cuff patch 140 may be supplied to be customized during the operative procedure.

The rotator cuff patch 110, 140 can be formed of a material similar to the materials for the patches described above in relation to FIGS. 1-4. That is, generally the rotator cuff patch 110, 140 can be formed of a substantially unitary or single piece of material or may be formed of a plurality of layers of material that are laminated together. In addition, the particular materials may be any appropriate materials, which exemplary include collagen, such as intestinal collagen material. The material can be formed into the desired shapes and laminated or cross-linked to provide selected properties. In addition, a selected blank sheet of material can be cut or formed according to a pre-determined or known template for a selected individual to provide a substantially customized rotator cuff implant 110.

With relation to the laminated material, in regards to the patches described in FIGS. 1-4, and the rotator cuff patches 110, 140, the laminated material is generally substantially aligned or co-extensive, in at least one dimension, prior to and during the lamination procedure. With reference to FIG. 9A, an implant blank 150 generally includes a plurality of layers of a selected material 152. The material for the layers 152 may be any appropriate material, such as the previously discussed ICL. Each of the layers 152, including a selected or appropriate number, are generally positioned relative one another wherein each layer touches or is adjacent to at least one other layer. The layers 152 are stacked atop one another prior to any laminating or cross-linking process to form the blank 150.

The layers 152 are generally equivalent in least a first dimension X. Although it will be understood that the dimension X need not be exact for each of the plurality of the layers 152, it will be understood that the layers 152 are generally equivalent in the dimension X. Therefore, the blank 150 can be formed by placing a plurality of layers 152 substantially adjacent one another in laminating the layers together.

With reference to FIG. 9B, each of the layers 152 are also generally aligned or parallel to one another as defined by a longitudinal axis Z of each of the layers 152. As illustrated in FIG. 9B, an exemplary blank is illustrated including three layers 152a, 152b, and 152c. Each of the layers includes a corresponding longitudinal axis designated Za to relate to the layer 152a, Zb to layer 152b, and Zc to layer 152c. Each of the longitudinal axes Za, Zb, and Zc are substantially parallel prior to forming the blank stack 150. Therefore, each of the layers 152a, 152b, and 152c is substantially aligned during the lamination process and remains substantially aligned after the lamination process and in the final implant. Therefore, because each of the layers are generally coextensive in at least one dimension and generally aligned along a longitudinal or selected axis, the final implant may generally be no larger than the smallest of the layers that form the blank 150. Although the blank 150 may be generally irregular because of slight or minor variations in the size of the layers of the material, the variations are generally cut or removed to form the final implant 156 (FIG. 10).

The lamination process may differ, depending upon the selected implant, as described herein, but generally includes substantially adhering each of the plurality of the layers 152 to at least one adjacent layer 152. In addition, a cross-linking initiator may be provided to allow for a selected amount of cross-linking between the plurality of the layers 152 to provide a selected characteristic of the blank 150 or a final implant. In addition, the material of each of the layers 152 may be internally cross-linked.

With continuing reference to FIG. 9, either before or after the lamination process, a selected cut or final implant shape or cut line 154 may be determined. After the blank 150 has been formed, the cut line can be used to cut the selected implant cross-section to form the final implant 156. As illustrated in FIG. 10, the final implant 156 may exemplary resemble the generally T-shaped implant as described in relation to the implant 10 of FIG. 1. It will be understood, however, that the cut lines or design of the blank 150 may be any appropriate shape to form any appropriate implant.

The final implant 156 includes a plurality of layers 152 of the material, which forms the implant 156 to provide various characteristics to the implant 156. For example, providing a laminated structure may include increased strength or rigidity to the final implant 156. In addition, it will be understood that the material of each of the plurality of the layers 152 need not necessarily be substantially identical to each of the other layers 152. That is, that one or more of the layers 152 may include a material that is not substantially similar to each of the other of the plurality of layers 152.

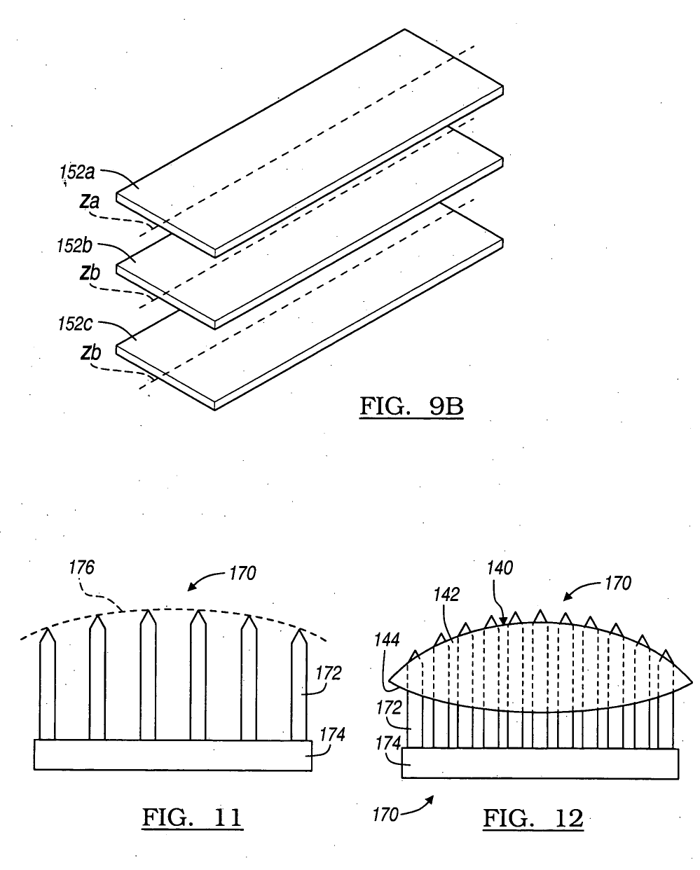

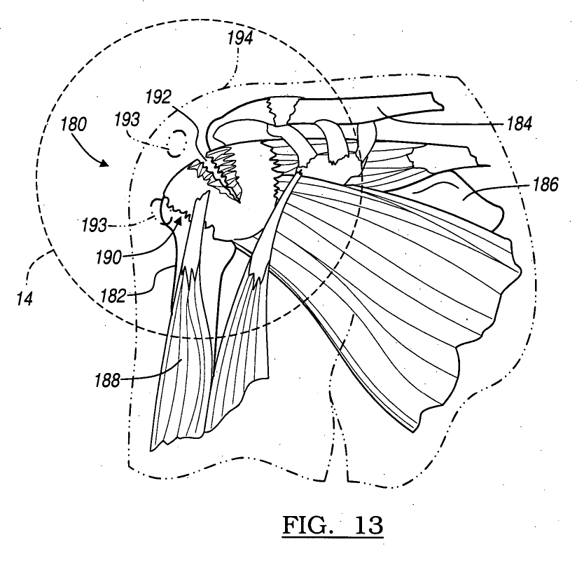

Once the blank 150 of the plurality of the layers of the material 152 is formed, it may also be shaped, either before, during, or after the lamination process, into a selected shape. For example, a selected jig or shaper 170 may be used to shape a selected implant design. With reference to FIG. 11, the jig 170 may include a plurality of piercing members or pins 172 that are mounted in an appropriate shape or form to a platform 174. The plurality of pins 172 may generally define a shape or contour 176 that may substantially mimic a selected shape of an implant. For example, the jig 170 may be used to form the substantially constant radius rotator cuff patch 140, as illustrated in FIG. 8.

With reference to FIG. 12, the jig 170 may include a plurality of pins 172 that substantially mimic a contour that is desired to be formed for the constant radius rotator cuff patch 140. The blank laminate 150 can be placed over the jig 170 and the pins 172 hold or may pass through a portion of the substantially constant radius rotator cuff patch 140. The pins 172 may pass through the material that form the rotator cuff patch 140 to substantially form pores within the rotator cuff patch 140. It will be understood that a plurality of the pins 172 may be positioned on the jig 170 to define a three-dimensional shape. The shape defined by the jig 170 allows for a substantial molding of the rotator cuff patch 140 once the blank 150 is placed upon the jig 170. The jig 170 may be used to hold the blank 150 during any processing of the material, such as laminating or cross-linking, to form the substantially constant radius rotator cuff patch 140.

For example, once the blank 150 is placed upon the jig 170, the lamination process may occur. The lamination of the blank forms a laminated construct, which may be the final implant or may be later worked to form the final implant. During the lamination process, the pores formed by the pins 170 may be used to or may provide for a means to substantially infiltrate the blank 150 with a laminating material to substantially form a laminated construct for the selected implant.

Also, the pores formed by the pins 172 in the substantially constant radius rotator cuff patch 140 may allow cellular infiltration after implantation. Although the holes may be formed after forming the patch 140 using generally known punching or drilling techniques. It will be understood, however, that the jig 170 or a jig similar to the jig 170 may be used to form any appropriate implant. Therefore, the benefits of providing the pores through the implant may be realized in any appropriate implant shape or form. Nevertheless, it will be understood that the pores formed by the pins 172 are not substantially required and may not be formed in the implant. That is, any appropriate jig or mold may be used to form a selected shape of the implant if a particular shape is required prior to implantation. Therefore, a substantially solid mold may also be used to form the implant with no pores save for any natural porosity.

In any case, any appropriate mold may be used to form the graft in a selected shape. For example, a generally oblong or tear-drop shaped mold may be used to form the rotator cuff patch 110. Similarly, an appropriate mold may be used to assist in the formation of the generally T-shaped cross-section implant 10, as illustrated in FIG. 1. In either case, a minimal amount of cutting may then be required to substantially customize the implant for a selected patient. Rather, the formation process, using the selected mold or other means, can be used to substantially fabricate the graft in a selected shape. Therefore, the elimination of interoperative or preoperative cutting of the graft can allow for a substantially easier implant of the graft to a selected site and reduce time and stress on a surgeon, during the operative procedure.

In addition to the pin jig 170 or any other appropriate mold, the patch 140, or any other appropriate implant shape, may be formed over a selected three dimensional structure. For example, the material may be placed over a stint shape such that the stent shape provides a structure or scaffold for forming the implant. The stent structure, or any appropriate structure, provides an artificial scaffold structure that the material of the implant can be positioned over before implanting. Therefore, the scaffold and the implant material are substantially biologically compatible to allow for ease of implantation.

The implant may still contain the various features, such as including a three dimensional shape, a substantially layered structure and other appropriate and above-described features. In addition, the implant may be implanted according to any appropriate method, such as that described below. Nevertheless, the implant includes an internal artificial scaffold structure to define a three dimensional shape. Therefore, the implant and the scaffold structure assists in holding the implant in a selected shape for a selected implantation. For example, the stent may be implanted into a capillary, or other appropriate tubular shape in the body to achieve selected results.

The various patches, including the soft tissue patches and the rotator cuff patches 110, 140, can be implanted in any of an appropriate manner depending upon the various procedures, the individual patient, and the desires of the physician. Nevertheless, the patches can be implanted using various arthroscopic or less invasive techniques than requiring a substantially open procedure. For example, the patches may be passed through a small incision or through a sleeve or tube that is positioned relative to the area in which the patch is required. The following exemplary procedures are not intended to limit the scope of the present disclosure or claims, but are merely intended to provide an example of various techniques that may be used to implant the selected patch.

With reference to FIG. 12, a shoulder area 180 may include various bony portions, such as a humerus and humeral head 182, clavicle 184, and scapula 184. In addition, various muscles may interconnect with various bone portions and generally referenced as muscle or soft tissue 188. The rotator cuff 190 is generally defined as the soft tissue surrounding the head of the humerus as it interacts with the glenoid to allow for articulation of the shoulder. The rotator cuff 190 may generate a tear or injury 192 due to age, trauma, or other actions that may cause the rotator cuff or the soft tissue of the rotator cuff 190 to tear or become injured. When such a tear does not or will not repair itself without intervention, surgical intervention may be required. The surgical intervention would generally be subcutaneous, such that a portal 193 may generally be formed through the soft tissue or dermis 194 that surrounds the shoulder 180.

One or a plurality of portals or incisions 193 are generally provided through the dermis 194 near the rotator cuff 190. The portals 196 allow for access to the rotator cuff 190 using any one or a plurality of instruments. For example, an arthroscope 198 may be provided through a first or a selected of the portals 196 while a tube or sleeve 200 is provided through a second.

Once it is determined that a selected patch is necessary, the patch material may be passed through the sleeve 200 in a properly deformed manner. For example, as described above, the rotator cuff patch 110 may be selectively cross-linked, such that a certain amount of moldability is allowed in the rotator cuff patch 110. Therefore, the rotator cuff patch 110 can be rolled or otherwise deformed to pass through the sleeve 200 adjacent to the tear 192. In this way, the rotator cuff patch 110 can be provided to the tear 192 in the rotator cuff 190 without providing a substantially large or overly traumatizing incision through the soft tissue or dermis 194.

An exemplary method of implanting a selected cuff patch 110 may be performed as generally described below. As discussed above, the selected area to which the cuff implant 110 is to be provided may provide or be used to create a template which is used to form or assist in the formation of the cuff patch 110. Once the template is designed, the template may be used to cut or form the cuff patch 110 to substantially match or mimic the template. Once the cuff patch 110 has been formed to substantially match the template, in both size, shape, etc. and is formed to include selected characteristics, such as moldability and strength, the cuff patch 110 may be provided for the procedure.

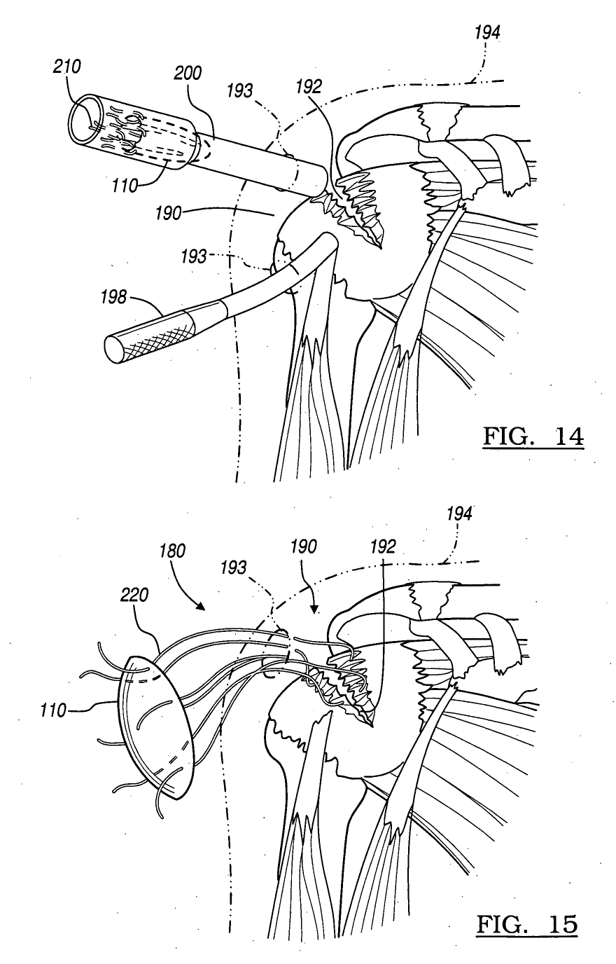

According to various embodiments, a suture 210 may be passed through the cuff patch 110 in various or selected positions. With reference to FIG. 14, the suture 210 or a plurality of sutures 210 may be passed through selected portions of the cuff patch 110 prior to providing the cuff patch 110 into the sleeve 200. In this way, the suture 210 is provided relative to the cuff patch 110 in the selected locations to allow for a substantially selected orientation and securability of the cuff patch 110 relative to the rotator cuff 190. Also, the cuff patch 110 may include a number of selected bores or apertures to receive or allow the suture 210 to pass through the patch 110. Once the sutures 210 are passed through the cuff patch 110, the cuff patch may then be positioned in the sleeve 200 and passed through the portal 196 to be positioned relative to the tear 192 in the rotator cuff 190.

Once the rotator cuff patch 110 has been positioned relative to the rotator cuff tear 192, the sutures 210 may be sutured or tied to the tissue of the rotator cuff 190 relative to the tear 192. Therefore, the suture 210 is provided already positioned through the rotator cuff patch 110 prior to its insertion through the dermis 194. Then the physician need only pass the sutures 210 through the soft tissue of the rotator cuff 190 and further tie the sutures to secure the rotator cuff patch 110 to the rotator cuff 190. Thus, the rotator cuff patch 110 can be used to patch the rotator cuff 190 through a less invasive procedure, not requiring a substantially large portal. Generally, the portals 196 may be about 0.5 cm to about 3 cm in length.

Although the rotator cuff patch 110 is described to pass through the sleeve 200, it will be understood that the rotator cuff patch 110, including the sutures 210, may be provided simply through the portal 196. That is, the sleeve 200 is not necessary and simply may allow for assistance in guiding the rotator cuff patch 110 to a selected position. Also, once the rotator cuff patch 110 is provided to the selected position, the sleeve 200, if used, can be removed from the portal 196 to allow for other instruments to be passed through the portal 196 to assist in positioning and fixing the rotator cuff patch 110 to the rotator cuff 190. For example, a suture tying instrument may be passed through the portal 196 to assist in tying the sutures 210 to the rotator cuff 190.

A further example of the method of implanting and affixing the rotator cuff patch 110 to a rotator cuff 190 will also be described. It will be understood that this exemplary description is also not intended to limit the scope of the disclosure or the appended claims. According to various embodiments, illustrated in FIG. 14, a suture 220 may first be tied to the rotator cuff 190. The suture 220 may be tied to the rotator cuff 190 and passed through the portals 196. The suture 220, or a plurality of the sutures 220, may be passed through selected portions of the rotator cuff 190 relative to the tear 192. Selected portions of the sutures 220 may then be passed through the portal 196 to engage the rotator cuff patch 110.

After the rotator cuff patch 110 has been formed, the sutures 220 engage the rotator cuff patch 110, such that the rotator cuff patch 110 can be fixed to the rotator cuff 190 relative to the tear 192. According to the exemplary embodiments the sutures 220 are first fixed to the rotator cuff 190 relative to the tear 192.

The rotator cuff patch 110 may then be passed over the sutures 220 to engage the rotator cuff 190 relative to the tear 192. The rotator cuff patch 110 may be passed over the sutures 220 according to commonly known techniques, such as those similar to a knot passing procedure. Therefore, a knot may be formed in the sutures 220 on the exterior of the dermis 194, such that the knots are passed with the rotator cuff patch 110 through the portal 196 in a selected manner. For example, as described above, the rotator cuff patch 110 may be generally moldable, such that even if it is larger than the portal 196 it may be deformed to pass through the portal 196. Therefore, the rotator cuff patch 110 may be passed through the portal 196 with knots that have been formed in the sutures 220, such that only the knots need to be tightened to fix the rotator cuff patch 110 relative to the tear 192. It will be understood that other portals 196 may be formed through the dermis 194 to allow for ease of viewing of the procedure. In addition, other portals may be provided to allow for instruments to be positioned relative to the tear 192 to the dermis 194. Nevertheless, the rotator cuff patch 110 may be provided through the selected portal 196, to repair the tear 192 and the rotator cuff 190.

Therefore, it will be understood that patches according to various design, sizes, and shapes can be formed using a plurality of materials. Generally the material may be laminated for selected characteristics, such as strength. The laminated material is generally co-extensive and can be cut or molded into a selected shape. In addition, the material may be cross-linked a selected amount to achieve a selected characteristic, such as moldability or rigidity. Nevertheless, the selected implant, formed from the laminated material can be used for a plurality of purposes or techniques, such as a rotator cuff patch or a reinforcement patch form a soft tissue harvest site. The patch may be substantially formed in a customized manner to fit the selected position or may be moldable to allow for on-site customization for the particular individual. It will also be understood that various exemplary embodiments are described in relation to specific exemplary procedures. Such description is not intended to limit the scope thereof, as each or all implants may be implanted using any appropriate procedure.

The description is merely exemplary in nature and, thus, variations that do not depart from the gist thereof are intended to be within the scope thereof. Such variations are not to be regarded as a departure from the spirit and scope of the description of the following claims.

What is claimed is:

1. An implant for use in repairing or reinforcing a tissue of a body, the implant comprising:
a structure defined by at least a first layer and a second layer;
said first layer and said second layer being generally held relative to one another in a generally aligned manner; and
an engagement portion defined by at least a portion of said laminated structure such that said laminated structure is able to be held relative to a selected portion of the tissue;
wherein said laminated structure is able to engage the tissue to assist in holding said tissue in a selected orientation.

2. The implant of claim 1, wherein said engagement portion includes at least one of an aperture and a core defined by said laminated structure.

3. The implant of claim 1, further comprising an engagement member, wherein said engagement portion includes at least one region defined by said laminated structure to allow for a substantially easy passing of said engagement member, wherein said engagement member is operable to interconnect said laminated structure and the tissue.

4. The implant of claim 1, wherein said laminated structure is defined by a number of layers in excess of said first layer and said second layer;
wherein each of said layers is laminated together to form a laminated construct implant for implantation including selected properties.

5. The implant of claim 4, wherein said selected properties include at least one of a strength, a rigidity, a size, a shape, a cross-sectional orientation, and combinations thereof.

6. The implant of claim 1, wherein said first layer and said second layer are formed of a material that is absorbable into the body after a selected period of time.

7. The implant of claim 6, wherein said bio-absorbable material includes collagen provided from a donor.

8. The implant of claim 1, wherein said first layer and said second layer are cross-linked to a selected degree to provide a selected property of the laminated structure prior to implant.

9. The implant of claim 1, wherein said generally aligned manner includes:
said first layer and said second layer each defining a longitudinal axis, and each axis is generally aligned with the other prior to and generally after lamination of the first layer and the second layer to form said laminated structure.

10. The implant of claim 1, wherein said first layer and said second layer include a selected dimension that is generally equivalent to each other, wherein said implant includes at least one dimension no larger than said generally equivalent dimension of said first and second layer.

11. The implant of claim 1, wherein said structure defines at least one:
a substantially “T” shaped cross-section formed along a plane generally transverse to a longitudinal axis of the structure;
a perimeter of said structure that includes an elongated portion that terminate in enlarged ends;
a dome including a substantially circular perimeter; and
a dome including a generally oblong shape wherein said dome defines a first radius and a second radius wherein said first radius differs from said second radius.

12. A method of making an implant comprising:
providing a first layer of material defining a first dimension;
positioning a second layer of material defining a second dimension relative to said first layer of material;
providing said first layer and said second layer such that said first dimension and said second dimension are generally co-extensive; and
laminating said first layer to said second layer to form a laminated construct from which the implant is able to be formed.
wherein positioning said second layer relative to said first layer includes substantially aligning a longitudinal axis of said second layer with a longitudinal axis of said first layer.

13. The method claim 12, further comprising:
cross-linking said first layer and said second layer to a selected degree wherein said cross-linking provides a selected property of the implant.

14. The method of claim 13, wherein said cross-linking said first layer and said second layer includes both intra-layer cross-linking and inter-layer cross-linking such that cross-links may be formed within each of the layers and between each of the layers.

15. The method of claim 12, further comprising:
positioning at least a third layer adjacent said first layer or said second layer prior to laminating said first layer or said second layer;
wherein positioning said first layer, said second layer, and said third layer forms a blank which is laminated in said laminating;
wherein each of said first layer, second layer and third layer are laminated to an adjacent layer to substantially form the implant.

16. The method of claim 15, further comprising:
positioning any number of layers relative to said first layer and said second layer and laminating each of the plurality of layers to at least one layer adjacent each layer to form a laminated construct to form the implant.

17. The method of claim 12, further comprising:
forming the implant from the laminated construct to include at least one of a selected size, shape, property, engagement portion and combinations thereof.

18. The method of claim 17, wherein forming the implant includes cutting a portion of the laminated structure such that after each of the selected cuts are made a resultant implant is formed that may be positioned relative to the soft tissue.

19. The method of claim 17, wherein forming the implant includes:
determining the selected position of the implant relative to the soft tissue;
creating a template that is able to generally conform with the soft tissue to provide a selected result; and
cutting said laminated construct using said template as a guide.

20. A method of forming an implant for implanting into a body, the method comprising:
selecting an implant characteristic including a generally three-dimensional contour;
forming a mold to generally define said generally three-dimensional contour;
positioning an implant material relative to said mold; and
curing said implant material to generally conform to said mold;
wherein said implant material includes a first layer and a second layer;
wherein said curing includes lamination.

21. The method of claim 20, further comprising:
positioning at least a first layer and a second layer relative to the mold, wherein said mold positions said at least said first layer and said second layer in a selected position; and
laminating said at least first layer and said second layer while said first layer and said second layer are positioned on said mold such that said laminated construct is formed to generally compliment said mold.

22. The method of claim 20, wherein said mold includes at least a piercing member able to pierce at least a portion of one of a first layer and a second layer to provide a selected aperture in said first layer or said second layer.

23. The method of claim 22, wherein said mold is able to provide a pore in said implant material in a substantially selected orientation or position.

24. The method of claim 22, further comprising:
providing a plurality of said projections to form a plurality of pores in said implant material;
wherein said pores are able to allow a selected material to enter said implant material.

25. The method of claim 20, wherein selecting an implant characteristic includes at least one of determining a contour to generally nestingly engage a rotator cuff and a contour to engage a condyle.

26. A method of providing an implant for a selected site in a soft tissue from which an autograft is taken, comprising:
selecting a position from which an autograft is to be harvested;
removing the autograft and forming a harvest site;
positioning an implant in said harvest site;
forming said implant including:
positioning a first layer relative to a second layer; and
laminating said first layer and said second layer to form a generally laminated construct;
wherein said first layer and said second layer are generally of equivalent in size prior to said laminating.

27. The method of claim 26, wherein selecting the position from which the autograft is to be taken includes:
selecting an appropriate soft tissue portion that is able to be used in a selected autograft implant site;
wherein said soft tissue interconnects a first bone portion and a second bone portion.

28. The method of claim 26, wherein removing the autograft includes removing an autograft portion from the tissue, wherein the removal of the autograft portion generally results in defining of a gap in the tissue.

29. The method of claim 26, further comprising forming said implant to substantially compliment said repair site.

30. The method of claim 26, further comprising:
fixing said implant relative to said repair site.

31. The method of claim 26, wherein forming an implant includes:
forming an aperture in the laminated construct that allows a selected fixation member to pass through said laminated construct to substantially interact with the tissue at a selected position relative to said repair site.

32. The method of claim 31, further comprising cross-linking a selected portion of said first layer and said second layer to provide a selected property to said laminated construct.

33. The method of claim 26, wherein forming an implant includes at least one of:
positioning said first layer relative to said second layer such that said laminated construct substantially compliments said repair site; and
cutting said laminated construct to substantially compliment said repair site;
wherein said implant substantially compliments said repair site for a generally customized fit.

34. A method of implanting an implant relative to a selected tissue portion using a generally less invasive procedure, the method comprising:
forming the implant to include a selected dimension;
forming a portal to achieve access to the selected tissue portion;
passing said implant through said portal;
providing a suture through said implant prior to passing said implant through said portal; and
engaging said selected soft tissue portion with said suture.

35. The method of claim 34, wherein forming an implant includes:
positioning a blank laminate relative to a jig and laminating said material such that said blank laminate forms a shape that is substantially complimentary to the jig.

36. The method of claim 35, wherein said jig includes at least one piercing element that is able to pierce the material either before or during the lamination process to allow the formation of a bore in said implant.

37. The method of claim 34, further comprising: defining a structure in said implant that allows a selected portion of the soft tissue to grow into said implant over a selected period of time.

38. The method of claim 34, further comprising providing a sleeve through said portal;
passing said implant through said portal, wherein said implant is deformed from said selected dimension to pass through said portal and is able to re-obtain said selected dimension after passing through said sleeve.

39. The method of claim 34, further comprising:
tying said suture to the soft tissue after the implant has passed through said portal to generally fix said implant relative to the soft tissue.

40. The method of claim 34, wherein engaging said soft tissue portion with said suture occurs before passing said suture through the implant;
wherein after said suture is provided through said implant said implant is passed over said suture to a selected position relative to the soft tissue;
wherein a knot is passed over the suture to substantially hold the implant relative to the soft tissue.

41. The method of claim 40, wherein said knot and said implant are passed over said suture substantially concurrently.

42. The method of claim 34, wherein providing a suture through said implant includes passing said suture through a bore defined by the implant.

43. The method of claim 34, further comprising:
forming a bore in said implant to receive an anchor; and
anchoring the implant with the anchor to a portion of the soft tissue relative to said soft tissue area.

44. An implant for use in repairing or reinforcing a tissue of a body, the implant comprising:
a structure that has a discrete three-dimensional shape and has at least a first layer and a second layer;
said first layer substantially held relative to said second layer in a generally aligned manner; and
an engagement portion defined by at least a portion of said structure such that said structure is able to be held relative to a selected portion of the tissue;
wherein said structure is substantially manufactured with said discrete three-dimensional shape.

45. The implant of claim 44, wherein said discrete three dimensional shape is defined by an implantable substructure;
wherein said structure is overlaying said implantable substructure to maintain said discrete three dimensional shape.

46. The implant of claim 45, wherein said implantable substructure includes a stent.

47. The implant of claim 44, wherein said discrete three dimensional shape includes at least one of a dome, a T-shape, a tube, a condylar shape and combinations thereof.
